(19) United States
(12) Patent Application Publication
(10) Pub. No.: US 2020/0034796 A1
(43) Pub. Date: Jan. 30, 2020

(54) METHODS FOR MANAGING ONE OR MORE UNCORRELATED ELEMENTS IN DATA AND DEVICES THEREOF

(71) Applicant: Mitchell International, Inc., San Diego, CA (US)

(72) Inventors: Mary Cassol, San Diego, CA (US); Vicki Dunbar, San Diego, CA (US); Valerie Lindgren, San Diego, CA (US); Pamela Martinez, San Diego, CA (US)

(21) Appl. No.: 16/520,026

(22) Filed: Jul. 23, 2019

Related U.S. Application Data
(60) Provisional application No. 62/702,752, filed on Jul. 24, 2018.

(51) Int. Cl. G06Q 10/10; G16H 50/20; G16H 10/60; G06Q 40/08

(52) U.S. Cl. CPC G06Q 10/1057; G06Q 40/08; G16H 10/60; G16H 50/20

(57) ABSTRACT

A method, non-transitory computer readable medium, and apparatus that identifies one of a plurality of diagnostic mapping tables based on a diagnostic code associated with one of a plurality of data environment formats in an electronic claim. The diagnostic code associated with one of the plurality of data environment formats is correlated to at least one of a plurality of parts and laterality associated with another one of the plurality of data environment formats based on the identified one of the plurality of diagnostic code mapping tables. One of a plurality of assessment ratings is determined based on the diagnostic code the correlated one of the plurality of parts and the laterality, and a categorization table associated with the another one of the plurality of data environment formats. Execution of one of a plurality of actions on the electronic claim in response to the determined one of the plurality of assessment ratings for the diagnostic code is initiated.

This application claims the benefit of U.S. Provisional Patent Application Ser. No. 62/702,752, filed Jul. 24, 2018, which is hereby incorporated by reference in its entirety.

### FIELD

This technology generally relates to methods, non-transitory computer readable medium, and devices for managing one or more uncorrelated elements in data.

### BACKGROUND

Current estimates predict that the amount of available data is set to reach about 44 zettabytes by 2020. Additionally, in more and more environments, a variety of different types of applications are identifying and providing this available data in response to particular electronic requests and other operations. Often this accessed data has valuable information to assist with the particular electronic requests and other operations, but the accessed data is often uncorrelated and uncategorized for the particular environment. As a result, even though accessible, the provided data is difficult to process and manage and thus does not facilitate the efficient completion of the particular electronic requests and other operations.

By way of example only, in the insurance industry an electronic claim may be received for processing of a claim for physical therapy treatment that includes a diagnosis code: M43.06 Spondylolysis, lumbar region. Spondylolysis is a crack or stress fracture of the vertebrae. This is a condition that most often occurs in children or athletes who participate in sports that involve repeated stress on the back. Unfortunately, in the auto casualty insurance environment, computing devices to assist with the electronic processing of invoices are unable for example to manage how to process this diagnostic code data to determine whether this diagnosis is related to a motor vehicle accident or not in an electronic claim. As a result, prior computing devices in this automated insurance claims processing environment may, for example, require operator input to manually evaluate the diagnostic code to make a determination which is time consuming and expensive or may incorrectly approve or deny the claim as part of the automated processing without proper evaluation of the available diagnostic code data.

Further by way of example only, these prior computing devices in this automated insurance claims processing environment are unable to properly categorize the available data. Currently, all existing severity of injury scales: Abbreviated Injury Scale; Organ Injury Scales; Injury Severity Score; New Injury Severity Score; and International Classification of Diseases (ICD) Injury Severity Score, have an emergency room focus. In other words, these scales were developed to assess morbidity and mortality in an emergency and are not correlated with data in other environments. Unfortunately, there are no severity of injury scales for ICD diagnosis codes that are specific to the auto casualty or workers compensation insurance industry complicating the efficient processing of electronic claims.

### SUMMARY

A method identifies, by a computing apparatus, one of a plurality of diagnostic mapping tables based on a diagnostic code associated with one of a plurality of data environment formats in an electronic claim. The diagnostic code associated with one of the plurality of data environment formats is correlated, by the computing apparatus, to at least one of a plurality of parts and laterality associated with another one of the plurality of data environment formats based on the identified one of the plurality of diagnostic code mapping tables. One of a plurality of assessment ratings is determined, by the computing apparatus, based on the diagnostic code the correlated one of the plurality of parts and the laterality, and a categorization table associated with the another one of the plurality of data environment formats. Execution of one of a plurality of actions on the electronic claim in response to the determined one of the plurality of assessment ratings for the diagnostic code is initiated by the computing apparatus.

A non-transitory computer readable medium having stored thereon instructions comprising executable code which when executed by one or more processors, causes the one or more processors to identify one of a plurality of diagnostic mapping tables based on a diagnostic code associated with one of a plurality of data environment formats in an electronic claim. The diagnostic code associated with one of the plurality of data environment formats is correlated to at least one of a plurality of parts and laterality associated with another one of the plurality of data environment formats based on the identified one of the plurality of diagnostic code mapping tables. One of a plurality of assessment ratings is determined based on the diagnostic code the correlated one of the plurality of parts and the laterality, and a categorization table associated with the another one of the plurality of data environment formats. Execution of one of a plurality of actions on the electronic claim in response to the determined one of the plurality of assessment ratings for the diagnostic code is initiated.

A computing apparatus includes a memory coupled to a processor which is configured to be capable of executing programmed instructions stored in the memory to identify one of a plurality of diagnostic mapping tables based on a diagnostic code associated with one of a plurality of data environment formats in an electronic claim. The diagnostic code associated with one of the plurality of data environment formats is correlated to at least one of a plurality of parts and laterality associated with another one of the plurality of data environment formats based on the identified one of the plurality of diagnostic code mapping tables. One of a plurality of assessment ratings is determined based on the diagnostic code the correlated one of the plurality of parts and the laterality, and a categorization table associated with the another one of the plurality of data environment formats. Execution of one of a plurality of actions on the electronic claim in response to the determined one of the plurality of assessment ratings for the diagnostic code is initiated.

This technology provides a number of advantages including providing methods, non-transitory computer readable medium, and devices for more effective and efficient managing one or more uncorrelated elements in data in different data environment formats for automated electronic claims processing. Additionally, this technology provides a clinically accurate diagnosis code to body part mapping including determination of laterality (side of body) for electronic third party auto bill review software. Further, this technology is able to accurately categorize and assign a severity of injury to a diagnosis code for electronic claims processing. Even further, this technology is able to translate this mapping and categorizations between different types of electronic bill review software which was not previously possible with prior software solutions.

### BRIEF DESCRIPTION OF THE DRAWINGS

FIG. 1 is a block diagram of an environment with an example of a computing apparatus that maps and categorizes one or more uncorrelated elements in data for automated electronic claims processing;

FIG. 2 is a block diagram of the example of the computing apparatus shown in FIG. 1;

FIG. 3 is a flow chart of an example of a method for managing one or more uncorrelated elements in data for automated electronic claims processing;

FIG. 4 is a table of an example of severity of injury categories for correlation with diagnostic code data;

FIG. 5 is a table of an example of a body part codes and corresponding descriptions; and

FIG. 6 is a table of an example of laterality identification codes and corresponding narratives.

### DETAILED DESCRIPTION

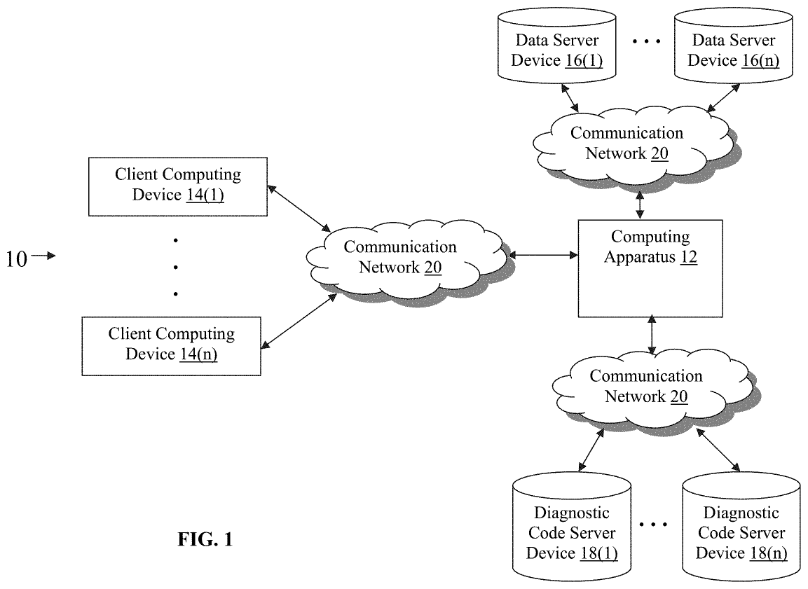

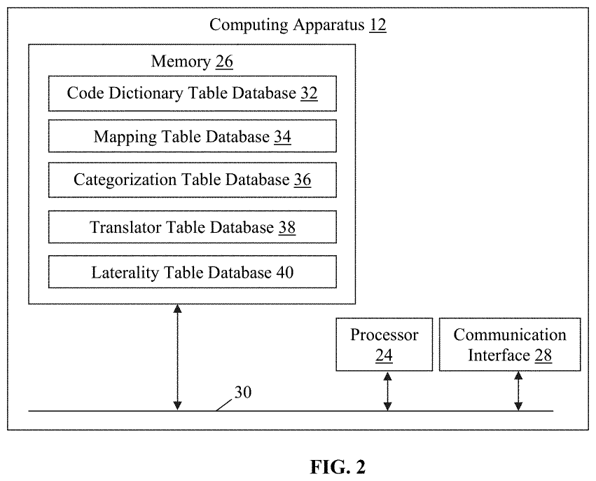

An environment 10 with an example of a computing apparatus 12 that maps and categorizes one or more uncorrelated elements in data for automated electronic claims processing is illustrated in FIGS. 1-2. In this particular example, the environment 10 includes the computing apparatus 12, client computing devices 14(1)-14(n), data server devices 16(1)-16(n), and diagnostic code server devices 18(1)-18(n) coupled via one or more communication networks 20, although the environment could include other types and numbers of systems, devices, components, and/or other elements as is generally known in the art and will not be illustrated or described herein. This technology provides a number of advantages including providing methods, non-transitory computer readable medium, and apparatuses for more effective and efficient managing one or more uncorrelated elements in different data environment formats for automated electronic claims processing.

Referring more specifically to FIGS. 1-2, the computing apparatus 12 is programmed to map and categorize one or more uncorrelated elements in data as illustrated and described herein, although the apparatus can perform other types and/or numbers of functions or other operations and this technology can be utilized with other types of claims. In this particular example, the computing apparatus 12 includes a processor 24, a memory 26, and a communication interface 28 which are coupled together by a bus 30, although the computing apparatus 12 may include other types and/or numbers of physical and/or virtual systems, devices, components, and/or other elements in other configurations.

The processor 24 in the computing apparatus 12 may execute one or more programmed instructions stored in the memory 26 to map and categorize one or more uncorrelated elements in data as illustrated and described in the examples herein, although other types and numbers of functions and/or other operation can be performed. The processor 24 in the computing apparatus 12 may include one or more central processing units and/or general purpose processors with one or more processing cores, for example.

The memory 26 in the computing apparatus 12 stores the programmed instructions and/or other data for one or more aspects of the present technology as described and illustrated herein, although some or all of the programmed instructions and/or data could be stored and/or executed or obtained elsewhere. A variety of different types of memory storage devices, such as a random access memory (RAM) or a read only memory (ROM) in the system or a floppy disk, hard disk, CD ROM, DVD ROM, or other computer readable medium which is read from and written to by a magnetic, optical, or other reading and writing system that is coupled to the processor 24, can be used for the memory 26. In this particular example, the memory 26 includes a code dictionary table database 32, a mapping table database 34, a categorization table database 36, and a translator table database 38, although the memory 26 can comprise other types and/or numbers of other modules, programmed instructions and/or data.

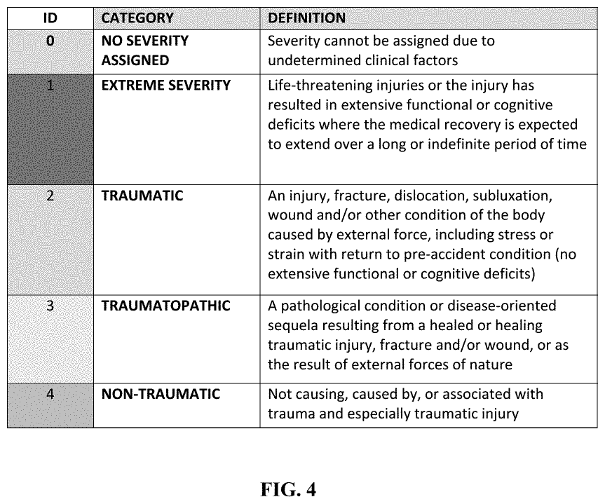

The code dictionary table database 32 may include diagnostic codes, such as ICD-9-CM diagnosis codes and ICD-10-CM diagnosis codes by way of example only, although other types and/or numbers of other codes or other designators in other types of industries or environments with one or more uncorrelated elements in data may be used, such as include ICD-11-CM diagnosis codes by way of example. The mapping table database 34 may include a stored mapping of different formats of diagnosis codes to body part mapping that may also include a laterality (side of body) assignment, although other types and/or number of other correlating mechanisms may be used. The categorization table database 36 may include a correlation of diagnosis codes to a severity of injury for each of the different formats which in this example is specific to and uniquely customized for the auto casualty or workers compensation insurance industry. By way of example, the categorization table database 36 for categorizing ICD diagnosis codes into one of five categories is illustrated in FIG. 4. In this particular example, the categories may comprise: 0—NO SEVERITY ASSIGNED—Severity cannot be assigned due to undetermined clinical factors; 1—EXTREME SEVERITY—Life-threatening injuries or the injury has resulted in extensive functional or cognitive deficits where the medical recovery is expected to extend over a long or indefinite period of time; 2—TRAUMATIC—An injury, fracture, wound and/or other condition of the body caused by external force, including stress or strain with return to pre-accident condition (no extensive functional or cognitive deficits); 3—TRAUMATOPATHIC—A pathological condition or disease-oriented sequela resulting from a healed or healing traumatic injury, fracture and/or wound, or as the result of external forces of nature; and 4—NON-TRAUMATIC—Not causing, caused by, or associated with trauma and especially traumatic injury, although other types and/or numbers of other categorizations or other ratings may be used. The translator table database 38 may, for example, provide a translation from a mapping in a workers compensation format to a mapping in an auto casualty industry format, although other types and/or numbers of cross translation techniques between other formats may be used. The laterality table database 40 may, for example, provide laterality identification, such as not applicable, left side, right side, bilateral and unilateral by way of example only, although other types and/or amounts of laterality identifications may be used Further examples of the programmed instructions and/or data in the code dictionary table database 32, the mapping table database 34, the categorization table database 36, the translator table database 38, and the laterality table database 40 are illustrated and described by way of the examples herein.

The communication interface 28 in the computing apparatus 12 operatively couples and communicates between one or more of the client computing devices 14(1)-14(n), the data server devices 16(1)-16(n), and the diagnostic code server devices 18(1)-18(n), which are all coupled together by one or more of the communication networks 20, although other types and numbers of communication networks or systems with other types and numbers of connections and configurations to other devices and elements. By way of example only, the communication networks 20 can use TCP/IP over Ethernet and industry-standard protocols, including NFS, CIFS, SOAP, XML, LDAP, SCSI, and SNMP, although other types and numbers of communication networks, can be used. The communication networks 20 in this example may employ any suitable interface mechanisms and network communication technologies, including, for example, any local area network, any wide area network (e.g., Internet), teletraffic in any suitable form (e.g., voice, modem, and the like), Public Switched Telephone Network (PSTNs), Ethernet-based Packet Data Networks (PDNs), and any combinations thereof and the like.

Each of the client computing devices 14(1)-14(n) may request unprocessed electronic claims, such as auto casualty electronic claims or workers compensation electronic claims by way of example only, from the computing apparatus 12 which may retrieve from a corresponding one of the data server devices 18(1)-18(n), although the data can be obtained in other manners and/or from other sources. Each of the client computing devices 14(1)-14(n) may request other types of data and/or instructions and may perform other types and/or numbers of other functions and/or operations.

Each of the data server devices 16(1)-16(n) may manage and store unprocessed electronic claims, such as auto casualty electronic claims or workers compensation electronic claims by way of example only, although each of the data server devices may store other types and/or amounts of programmed instructions and/or data. Additionally, each of the diagnostic code server devices 18(1)-18(n) may store and provide requested information and/or other content about diagnostic codes, such as ICD codes by way of example only, although each of the diagnostic code server devices may store other types and/or amounts of programmed instructions and/or data. The computing apparatus 12 may interact with each of the data server devices 16(1)-16(n) and/or each of the diagnostic code server devices 18(1)-18(n) via one or more of the communication networks 20, for example, although other types and/or numbers of storage media in other configurations with other stored information could be used. Each of the data server devices 16(1)-16(n) and/or each of the diagnostic code server devices 18(1)-18(n) also may comprise various combinations and types of storage hardware and/or software and represent a system with multiple network server devices in a data storage pool, which may include internal or external networks. Various network processing applications, such as CIFS applications, NFS applications, HTTP Web Network server device applications, and/or FTP applications, may be operating on each of the data server devices 16(1)-16(n) and/or each of the diagnostic code server devices 18(1)-18(n) and may transmit data in response to requests from the computing apparatus 12.

Each of the client computing devices 14(1)-14(n), each of the data server devices 16(1)-16(n), and each of the diagnostic code server devices 18(1)-18(n) may include a processor, a memory, and a communication interface, which are coupled together by a bus or other link, although other type and/or numbers of other devices and/or nodes as well as other network elements could be used.

Although the exemplary network environment 10 with the computing apparatus 12, the client computing devices 14(1)-14(n), the data server devices 16(1)-16(n), the diagnostic code server devices 18(1)-18(n), and the communication networks 20 are described and illustrated herein, other types and numbers of systems, devices, components, and/or elements in other topologies can be used. It is to be understood that the systems of the examples described herein are for exemplary purposes, as many variations of the specific hardware and software used to implement the examples are possible, as will be appreciated by those skilled in the relevant art(s).

In addition, two or more computing systems or devices can be substituted for any one of the systems or devices in any example. Accordingly, principles and advantages of distributed processing, such as redundancy and replication also can be implemented, as desired, to increase the robustness and performance of the devices, apparatuses, and systems of the examples. The examples may also be implemented on computer system(s) that extend across any suitable network using any suitable interface mechanisms and traffic technologies, including by way of example only teletraffic in any suitable form (e.g., voice and modem), wireless traffic media, wireless traffic networks, cellular traffic networks, G3 traffic networks, Public Switched Telephone Network (PSTNs), Packet Data Networks (PDNs), the Internet, intranets, and combinations thereof.

The examples also may be embodied as a non-transitory computer readable medium having instructions stored thereon for one or more aspects of the present technology as described and illustrated by way of the examples herein, as described herein, which when executed by the processor, cause the processor to carry out the steps necessary to implement the methods of this technology as described and illustrated with the examples herein.

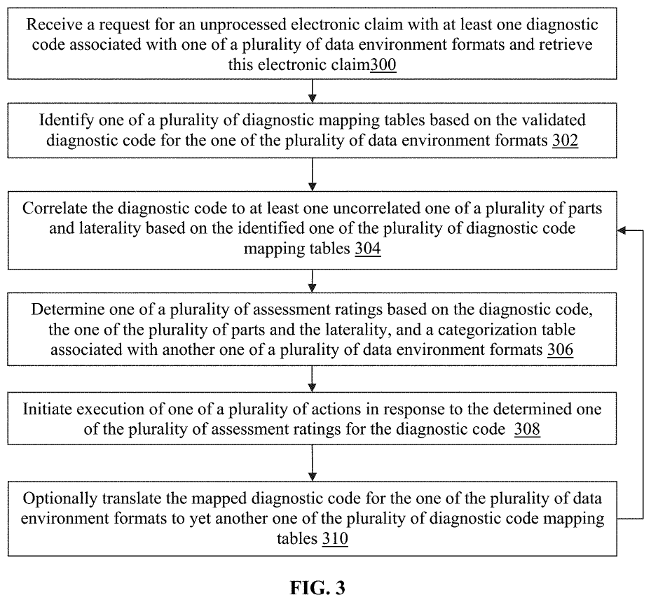

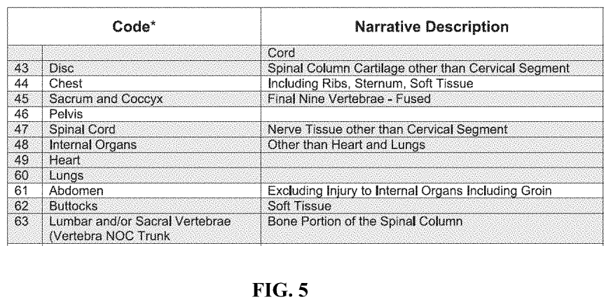

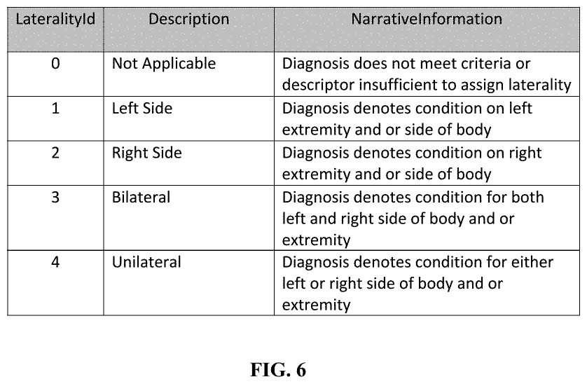

An example of a method for managing one or more uncorrelated elements in data will now be described with reference to FIGS. 1-6. Referring more specifically to FIG. 3, in step 300 the computing apparatus 12 may receive a request for an unprocessed electronic claim from one of the plurality of client computing devices 14(1)-14(n), such as a request for an unprocessed electronic workers compensation claim or an electronic third party auto casualty claim by way of example only. The electronic claim may have at least one diagnostic code associated with one of a plurality of data environment formats. Based on the received request, the computing apparatus 12 may retrieve this unprocessed electronic claim from one of the data server devices 16(1)-16(n) which may store electronic worker compensation claims or electronic auto casualty claims for processing by one of the client computing devices 14(1)-14(n) by way of example only, although other types and/or amounts of data may be stored. By way of a further example only, the electronic claim may be an electronic workers compensation claim for physical therapy treatment that includes a diagnosis code: M43.06 Spondylolysis, lumbar region.

The computing apparatus 12 may also determine if the at least one diagnostic code in the electronic claim is one of a plurality of valid diagnostic codes for the one of the plurality of data environment formats. In this example, the computing apparatus 12 may determine if the one of the ICD is one of a plurality of valid ICD diagnostic codes stored in this example in the code dictionary table database 32 for the one of the plurality of data environment formats, although the diagnostic code can be validated in other manners. If the at least one diagnostic code in the electronic claim is determined by the computing apparatus 12 to be invalid, then an electronic communication rejecting the electronic claim may be transmitted, although other types of actions may be taken. This external automated validation can streamline and prevent one of the client computing devices 14(1)-14(n) from working on an invalid electronic claim.

In step 302, the computing apparatus 12 may identify one of a plurality of diagnostic mapping tables stored in mapping table database 34, in this example, based on the validated diagnostic code for the one of the plurality of data environment formats. Each of the diagnostic mapping tables has one or more associated coding formats for another one of the plurality of data environment formats that can be correlated to the validated diagnostic code by the computing apparatus 12.

In step 304, the computing apparatus 12 may map the received diagnostic code for the one of the plurality of data environment formats in the retrieved claim data to a numeric or other identifier for at least one of a plurality of parts, such as human body part data by way of example only, for another one of the plurality of data environment formats based on the identified one of the plurality of diagnostic code mapping tables. By way of example, the computing apparatus 12 may map the received diagnostic code M43.06 to the NcciBodyPartId numeric identifier ‘63’ which refers to a body part: the Vertebrae—Thoracic/Lumbar/Sacral region of the body using the example of a portion of a mapping table stored in the mapping table database 34 as shown below:

| Diagnosis Code | Icd Version | Start Date | NcciBodyPartId |
|---|---|---|---|
| M43.06 | 10 | 2015-10-01 00;0 . . . | 63 |

In step 304, the computing apparatus 12 may also use the mapping table to determine a particular location or laterality, i.e. side of the body, of the identified body part or other region, which laterality data also may be from a different one of the plurality of data environment formats, although other types and/or other numbers of uncorrelated data elements in the other ones of the plurality of data environment formats may be correlated to the diagnostic code in the one of the plurality of data environment formats. In this example, each of the diagnostic codes may have a laterality indication stored in the stored mapping table from the database 34, although other manners for determining laterality may be used. By way of further example, the mapping table may provide a corresponding LateralityId numeric identifier ‘0’ which for this example indicates that laterality for diagnosis code M43.06 does not apply (i.e. diagnostic code does not meet criteria or is insufficient to indicate laterality). An illustration of a portion of a mapping table stored in the mapping table database 34 which illustrates this is provided below:

| Diagnosis | | | | | | | | | | Laterality |
|---|---|---|---|---|---|---|---|---|---|---|
| Code | Ic . . . | Star . . . | En . . . | No . . . | Tra . . . | D . . . | Description | Di . . . | Di . . . | Id |
| M43.06 | 10 | 2015 . . . | 2999 . . . | False | False | 0 | Spondylolysis, | Nu . . . | 4 | 0 |
| | | | | | | | lumbar region | | | |

In other examples, the laterality may be determined in other manners, such as with laterality information stored in laterality table database 40 that can be correlated to the identified body part and/or based on other information in the claim being processed by the computing apparatus.

In step 306, the computing apparatus 12 may determine one of a plurality of assessment ratings based on the validated diagnostic code, the one of the plurality of parts, the laterality and a categorization table for the another one of the plurality of data environment formats as illustrated in FIG. 4, although other types and/or numbers of factors may be to determine the assessment and/or other types of categorizations may be used. In the example above for the electronic workers compensation claim for physical therapy treatment, the computing apparatus 12 may determine that for the validated diagnosis code: M43.06 Spondylolysis, the part is the lumbar region and the laterality is none and based on the categorization table and information in the electronic claim associated with the diagnostic code that the assessment is a DiagnosisSeverityId rating assessment of 4-Non-traumatic (Not causing, caused by, or associated with trauma and especially traumatic injury). By way of example, the computing apparatus 12 may optionally use artificial intelligence engine that utilizes deep learning to store previously analyzed data related to corresponding diagnostic codes and may develop and refine an algorithm or other executable rule or rules to further assist with determining the assessment rating based on one or more of the factors discussed in the example above. An example of the results of this assessment are illustrated in a portion of a resulting table below:

| Diagnosis | | | | | | | | | Diagnosis | Laterality |
|---|---|---|---|---|---|---|---|---|---|---|
| Code | Ic . . . | Star . . . | En . . . | No . . . | Tra . . . | D . . . | Description | Di . . . | SeverityId . . . | Id |
| M43.06 | 10 | 2015 . . . | 2999 . . . | False | False | 0 | Spondylolysis, | Nu . . . | 4 | 0 |
| | | | | | | | lumbar region | | | |

With this determined assessment rating, the electronic claim can more quickly and accurately be processed by the computing apparatus 12 saving valuable time, eliminating the expense of an external review and an inaccurate payment or other disposition of the electronic claim.

In step 308, the computing apparatus 12 may initiate execution of one of a plurality of actions in response to the determined one of the plurality of assessment ratings for the diagnostic code. By way of example only, the computing apparatus 12 may initiate an action to transmit an acceptance or rejection of the electronic claim or to initiate an action to electronically request additional data or a further evaluation, although other types and/or numbers of actions may be initiated.

In the example being used herein, for the diagnosis code M43.06 where DiagnosisSeverityId is indicated as ‘4’ (non-traumatic), the computing apparatus 12 may deny reimbursement of the electronic claim as this diagnostic code has no possibility of accident relatedness. Accordingly, with this automated management of one or more uncorrelated elements in data, the claimed technology provides a much higher accuracy and consistency at a faster rate when electronic processing of claims. Based on the determined ratings assessment, if a diagnosis code has been assigned a value=3 (TRAUMATOPATHIC: A pathological condition or disease-oriented sequela resulting from a healed or healing traumatic injury, fracture and/or wound, or as the result of external forces of nature), an automated alert may be initiated to alert the user that review of medical records and/or billing may be warranted to confirm accident relatedness.

In step 310, the computing apparatus 12 may optionally translate the mapped diagnostic code for the one of the plurality of data environment formats to yet another one of the plurality of diagnostic code mapping tables, such as a translation from a workers compensation mapping to a third party auto casualty mapping, although other types of translations may be completed. By way of example, for the diagnosis code M43.06, NcciBodyPartId ‘63’ which refers to the Vertebrae—Thoracic/Lumbar/Sacral region of the body, once the NcciBodyPartId is identified as described earlier, the computing apparatus 12 may translate the NcciBodyPartId to a HybridBodyPartId that is specific to the auto casualty market via a translator table in the translator table database utilizing the determination and assessment from the prior evaluation. In this example, the HybridBodyPartId ‘4001’ referenced is the Upper Back (Thoracic) or ‘4003’ Lower Back (Lumbar—Coccyx) region of the body.

| Diagnosis Code | IcdVersion | StartDate | NcciBodyPartId |
|---|---|---|---|
| M43.06 | 10 | 2015-10-01 00:0 . . . | 63 |

| HybridBodyPa . . . | Description |
|---|---|
| 4001 | Upper Back (Thoracic) |
| 4003 | Lower Back (Lumbar - Coccyx) |

Accordingly, in this example the computing apparatus 12 may use the stored translation data table to translate this value (NcciBodyPartId=63) to an auto-casualty specific diagnosis body part mapping (HybridBodyPartId=4003) and then may for example optional proceed back to step 304 as described above to automatically process the diagnostic code in the electronic claim in the same manner as described in the example above, although the claim may be processed in other manners.

Thus, as illustrated and described by way of the examples herein, this technology provides more effective and efficient management of one or more uncorrelated elements in data in different data environment formats for automated electronic claims processing. Additionally, this technology provides an automated clinically accurate diagnosis code to body part mapping including determination of laterality (side of body) for electronic third party auto bill review software. Further, this technology is able to accurately categorize and assign a severity of injury to a diagnosis code for electronic claims processing. Even further, this technology is able to automatically translate this management of uncorrelated data between different types of electronic claim formats.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A method comprising:
identifying, by a computing apparatus, one of a plurality of diagnostic mapping tables based on a diagnostic code associated with one of a plurality of data environment formats in an electronic claim;
correlating, by the computing apparatus, the diagnostic code associated with one of the plurality of data environment formats to at least one of a plurality of parts and laterality associated with another one of the plurality of data environment formats based on the identified one of the plurality of diagnostic code mapping tables;
determining, by the computing apparatus, one of a plurality of assessment ratings based on the diagnostic code the correlated one of the plurality of parts and the laterality, and a categorization table associated with the another one of the plurality of data environment formats; and
initiating, by the computing apparatus, execution of one of a plurality of actions on the electronic claim in response to the determined one of the plurality of assessment ratings for the diagnostic code.

2. The method as set forth in claim 1 wherein the determining the one of the plurality of assessment ratings is further based on data in the electronic claim related to the diagnostic code.

3. The method as set forth in claim 1 further comprising:
determining, by the computing apparatus, when the diagnostic code in the received request is one of a plurality of valid diagnostic codes for the one of the plurality of data environment formats;
wherein the initiating execution of one of a plurality of actions on the electronic claim further comprises transmitting an electronic communication rejecting the electronic claim when the determination indicates the diagnostic code is invalid for the one of the plurality of data environment formats.

4. The method as set forth in claim 1 further comprising translating, by the computing apparatus, the diagnostic code to another one of the plurality of diagnostic mapping tables associated with yet another one of the plurality of data environment formats.

5. The method as set forth in claim 4 wherein the yet another one of the plurality of data environment formats is a workers compensation diagnostic format and the another one of the plurality of data environment formats is an auto casualty diagnostic mapping format.

6. The method as set forth in claim 1 wherein the electronic claim comprises an electronic auto casualty claim and the at least one diagnostic code is at least one ICD code.

7. A non-transitory computer readable medium having stored thereon instructions comprising executable code which when executed by one or more processors, causes the one or more processors to:
identify one of a plurality of diagnostic mapping tables based on a diagnostic code associated with one of a plurality of data environment formats in an electronic claim;
correlate the diagnostic code associated with one of the plurality of data environment formats to at least one of a plurality of parts and laterality associated with another one of the plurality of data environment formats based on the identified one of the plurality of diagnostic code mapping tables;
determine one of a plurality of assessment ratings based on the diagnostic code, the correlated one of the plurality of parts and the laterality, and a categorization table associated with the another one of the plurality of data environment formats; and
initiate execution of one of a plurality of actions on the electronic claim in response to the determined one of the plurality of assessment ratings for the diagnostic code.

8. The medium as set forth in claim 7 wherein the determine the one of the plurality of assessment ratings is further based on data in the electronic claim related to the diagnostic code.

9. The medium as set forth in claim 7 wherein the executable code when executed by the one or more processors further causes the one or more processors to:
determine when the diagnostic code in the received request is one of a plurality of valid diagnostic codes for the one of the plurality of data environment formats;
wherein the initiating execution of one of a plurality of actions on the electronic claim further comprises transmitting an electronic communication rejecting the electronic claim when the determination indicates the diagnostic code is invalid for the one of the plurality of data environment formats.

10. The medium as set forth in claim 7 wherein the executable code when executed by the one or more processors further causes the one or more processors to:
translate the diagnostic code to another one of the plurality of diagnostic mapping tables associated with yet another one of the plurality of data environment formats.

11. The medium as set forth in claim 10 wherein the yet another one of the plurality of data environment formats is a workers compensation diagnostic format and the another one of the plurality of data environment formats is an auto casualty diagnostic mapping format.

12. The medium as set forth in claim 7 wherein the electronic claim comprises an electronic auto casualty claim and the at least one diagnostic code is at least one ICD code.

13. A computing apparatus comprising:
a processor; and
a memory coupled to the processor which is configured to be capable of executing programmed instructions stored in the memory to:
identify one of a plurality of diagnostic mapping tables based on a diagnostic code associated with one of a plurality of data environment formats in an electronic claim;
correlate the diagnostic code associated with one of the plurality of data environment formats to at least one of a plurality of parts and laterality associated with another one of the plurality of data environment formats based on the identified one of the plurality of diagnostic code mapping tables;
determine one of a plurality of assessment ratings based on the diagnostic code, the correlated one of the plurality of parts and the laterality, and a categorization table associated with the another one of the plurality of data environment formats; and
initiate execution of one of a plurality of actions on the electronic claim in response to the determined one of the plurality of assessment ratings for the diagnostic code.

14. The apparatus as set forth in claim 13 wherein the determine the one of the plurality of assessment ratings is further based on data in the electronic claim related to the diagnostic code.

15. The apparatus as set forth in claim 13 wherein the executable code when executed by the one or more processors further causes the one or more processors to:
determine when the diagnostic code in the received request is one of a plurality of valid diagnostic codes for the one of the plurality of data environment formats;
wherein the initiating execution of one of a plurality of actions on the electronic claim further comprises transmitting an electronic communication rejecting the electronic claim when the determination indicates the diagnostic code is invalid for the one of the plurality of data environment formats.

16. The apparatus as set forth in claim 13 wherein the processor coupled to the memory is further configured to be capable of executing at least one additional programmed instruction stored in the memory to:
translate the diagnostic code to another one of the plurality of diagnostic mapping tables associated with yet another one of the plurality of data environment formats.

17. The apparatus as set forth in claim 16 wherein the yet another one of the plurality of data environment formats is a workers compensation diagnostic format and the another one of the plurality of data environment formats is an auto casualty diagnostic mapping format.

18. The apparatus as set forth in claim 13 wherein the electronic claim comprises an electronic auto casualty claim and the at least one diagnostic code is at least one ICD code.
